(19) United States
(12) Patent Application Publication
(10) Pub. No.: US 2022/0306550 A1
(43) Pub. Date: Sep. 29, 2022

(54) PROCESS FOR PREPARING ALKENES

(71) Applicants: thyssenkrupp Industrial Solutions AG, Essen (DE); thyssenkrupp AG, Essen (DE)

(72) Inventors: Nils TENHUMBERG, Dortmund (DE); Stefan GEHRMANN, Euskirchen (DE); Michael KLEIBER, Hattersheim (DE)

(73) Assignees: thyssenkrupp Industrial Solutions AG, Essen (DE); thyssenkrupp AG, Essen (DE)

(21) Appl. No.: 17/640,151

(22) PCT Filed: Aug. 13, 2020
(86) PCT No.: PCT/EP2020/072695

§ 371 Date: Mar. 3, 2022

(30) Foreign Application Priority Data
Sep. 5, 2019 (DE) 10 2019 213 494.7

(51) Int. Cl. C07C 1/04; C07C 29/151; C07C 1/24; C07C 7/11; C07C 29/86; B01J 21/18; B01J 23/75; B01J 35/00; B01D 53/14

(52) U.S. Cl. CPC C07C 1/0485; C07C 29/1516; C07C 1/24; C07C 7/11; C07C 29/86; B01J 21/18; B01J 23/75; B01J 35/006; B01J 35/0013; C07C 1/0435; B01D 53/1487; C07C 2521/18; C07C 2523/75

(57) ABSTRACT

A process can be used to prepare alkenes by catalytic conversion of synthesis gas to a first mixture comprising alkenes and alcohols. The alcohols present in the first mixture are converted to the corresponding alkenes by dehydration in a subsequent step. At least one alkene having two to four carbon atoms is obtained as isolated product from a product mixture by processing thereof and/or separation steps. In the catalytic conversion, a catalyst is preferably used that comprises grains of non-graphitic carbon having cobalt nanoparticles dispersed therein. The cobalt nanoparticles have an average diameter dₚ of 1-20 nm. An average distance D between individual cobalt nanoparticles in the grains is 2-150 nm. A combined total mass fraction ω of metal in the grains is from 30%-70% by weight of a total mass of the grains such that 4.5 dp/ω>D≥0.25 dp/ω.

The present invention relates to a process for preparing alkenes by catalytic conversion of synthesis gas to a first mixture comprising alkenes and alcohols, wherein alcohols present in this mixture are converted to the corresponding alkenes by dehydration in at least one subsequent step.

### PRIOR ART

The dehydration of alcohols to the corresponding alkenes is a well-known reaction for the preparation of alkenes and is used industrially, for example for the production of bioethene from bioethanol. The catalytic dehydration of ethanol to ethene is carried out on a silicon-aluminum catalyst at 315-400° C. and low pressures of up to 20 bar with high conversions and selectivities.

US 2009/0281362 A1 describes the catalytic dehydration of 1-propanol or an ethanol/propanol mixture at 160-270° C. (preferably between 200-225° C.) and 1-45 bar (preferably between 10 and 20 bar). Furthermore, the preparation of propanol or the alcohol mixture using a synthesis gas-based process is also described. The conversion of hydrocarbons to synthesis gas is mentioned, inter alia, as a source of synthesis gas. The formation of a product mixture of alcohols and alkenes is not mentioned, nor is the use of metallurgical gases as a source of synthesis gas. In the process described, C3+ alcohols are preferably removed before carrying out the dehydration, since these have a disadvantageous effect on the dehydration and result in an increased formation of alkanes. Ethers are mentioned as possible intermediates of the dehydration. Heteropolyacids such as 12-tungstophosphoric acid, 12-tungstosilicic acid, 18-tungstophosphoric acid and 18-tungstosilicic acid are used as catalysts.

1-Butene is obtained from the C4 raffinate or by dimerization of ethene. Moreover, numerous industrial processes for the dehydration of alcohols to alkenes are known from the prior art, in which different reaction conditions are used.

DD 257 740 A3 discloses a process for preparing C2-C4-alkenes in which an alcohol mixture comprising methanol and higher aliphatic alcohols is first produced by reacting synthesis gas on copper-containing catalysts, followed by dehydration to the alkenes by reaction of the higher aliphatic alcohols on zeolitic catalysts having a pentasil structure at temperatures of 250 to 600° C. and at pressures above 100 kPa. Alternatively, heteropolyacid-containing catalysts may be used in a mixture with dehydration catalysts such as alumina. When converting the synthesis gas on a copper-containing catalyst, a mixture is obtained in this known process which consists almost exclusively of alcohols, this mixture comprising 53% methanol, 17% ethanol and 18% water and only a comparatively small proportion of propanol and butanol.

DE 30 05 550 A1 describes a process for preparing alkenes by dehydration of aliphatic alcohols, in which an alcohol mixture comprising methanol and higher aliphatic alcohols is first produced from synthesis gas using a catalyst based on a copper oxide, zinc oxide, aluminum oxide or potassium oxide, which comprises chromium, cerium, lanthanum, manganese or thorium as promoter. Methanol is separated off from this alcohol mixture, and ethanol and the propanols are dehydrated on a dehydration catalyst to give the corresponding alkenes. The alkene mixture obtained is optionally fractionated.

U.S. Pat. No. 6,768,035 B2 describes a process in which synthesis gas is reacted on a cobalt catalyst in a Fischer-Tropsch reactor, with a liquid phase and a gas phase being formed and hydrocarbons being condensed from the gas phase. Water is separated off and the liquid waxy gas phase and the condensed gas phase are separated in a distillation into a light phase comprising methane and ethane, a C3-C4 stream comprising alkenes and into another stream also comprising propanol and butanol, the alkene-containing stream being dehydrated or isomerized on an acidic catalyst. After dehydration, water has to be removed and the liquid hydrocarbon-containing stream is redistilled wherein an alkene-containing stream is obtained comprising 2-butene and 1-butene. In this process, the focus is on the production of an alkylate having a high octane number, i.e. the primary concern is not the targeted production of individual alkenes and alcohols from the product mixture which is formed during the catalytic conversion of the synthesis gas. Rather, the alkene fraction is mixed with an isoalkane stream comprising isobutane and then reacted with an alkylation catalyst to form a branched isoalkane alkylate.

U.S. Pat. No. 8,129,436 B2 describes a process for producing an alcohol mixture from synthesis gas, a mixture of alcohols and oxygen-containing compounds being obtained. It is proposed to strip the product mixture with a methanol-containing stream in order to remove a proportion of the carbon dioxide and inert gases present in the product stream. In addition, dehydration can take place downstream in order to convert some of the ethanol formed, and optionally propanol, to the corresponding alkenes. Potassium-modified molybdenum sulfide catalysts are used in the conversion of the synthesis gas. This known process affords very complex product mixtures which do not contain alkenes but do contain relatively small amounts of alkanes and not only C2-C5 alcohols but in some cases also higher proportions of methanol and many other oxygen-containing compounds such as aldehydes, carboxylic acids, ketones, esters, ethers and also mercaptans and alkyl sulfides.

It is known from US 2007/0244348 A1 to produce an alcohol mixture from a synthesis gas stream obtained, for example, by steam reforming of natural gas, using a Fischer-Tropsch catalyst, which alcohol mixture mainly comprises methanol and ethanol, but only a few higher alcohols having 3 or more carbon atoms. The alcohol mixture is then reacted with a second catalyst to form an alkene product. The Fischer-Tropsch catalyst comprises, for example, cobalt oxide and is modified with copper oxide. For the conversion of the alcohols to the alkenes, zeolitic or other molecular sieves are used, particularly silicoaluminophosphates. In this known process, either an alcohol mixture having a high proportion of methanol (more than 50%) is formed in the first step during the catalytic conversion of the synthesis gas, or alkanes are formed, particularly methane and ethane, but no alkenes, in addition to the alcohols. The alkenes are thus only generated in a subsequent reaction from the previously formed alcohols.

The object of the present invention is to develop an improved process for preparing alkenes, especially having two to four carbon atoms, by catalytic conversion of synthesis gas, in which the complex product mixture of alcohols, alkenes and alkanes can be selectively converted to downstream products and thus a high quality product(s) for the fuel market and/or chemical industry can be produced. The object of the present invention is furthermore to provide a process of the aforementioned type in which the purification of the complex product mixture is facilitated.

The aforementioned object is achieved by a process for producing alkenes of the type specified at the outset which has the features of claim 1.

According to the invention, it is provided that at least one alkene having two to four carbon atoms is obtained as isolated product from the product mixture by processing thereof and/or separation steps, either before or after the step of dehydration of the alcohols.

In contrast to the prior art, according to the invention not only alcohols are produced in the catalytic conversion of synthesis gas, i.e. already in this first step, but already a mixture of alcohols and alkenes, which also comprises alkanes. The alcohols present in the mixture are then subsequently converted to further alkenes by dehydration. In this way, a higher proportion of alkenes can be obtained with comparatively little effort, wherein specific alkenes can then be isolated by specific processing and/or separation steps, which may be used, for example, as reactants for further syntheses in industrial processes.

According to the present invention, the synthesis of higher alcohols and C2-C5-alkenes from synthesis gas firstly comprises the provision of the synthesis gas, the catalytic synthesis of the higher alcohols from this synthesis gas (“higher alcohols” are understood here to mean alcohols having at least two carbon atoms) and the purification or separation of the product mixture. The provision of the synthesis gas optionally also includes the purification and conditioning of the synthesis gas in addition to the preparation of the synthesis gas. Fossil fuels such as natural gas, coal, but also CO-rich and CO₂-rich gases, for example from steel or cement works, and hydrogen, can be used as feed for the provision of the synthesis gas. It is also possible to obtain the synthesis gas used from biomass. The hydrogen is preferably produced in a sustainable manner and/or with low CO₂ emissions, for example by water electrolysis or methane pyrolysis. The electricity for operating the hydrogen production is preferably generated using renewable energies.

According to the invention, the catalytic synthesis of the higher alcohols from synthesis gas can be carried out, for example, at reaction temperatures of 200° C. to 360° C., preferably at temperatures of 220° C. to 340° C., more preferably at 240° C. to 320° C., especially at 260° C. to 300° C., for example at about 280° C. In addition, this reaction can be carried out at atmospheric pressure or at elevated pressure, for example at a reaction pressure of 10 bar to 110 bar, in particular at 30 bar to 90 bar, preferably at 50 bar to 70 bar, for example at about 60 bar.

The resulting product mixture of unreacted synthesis gas, alcohols, alkenes and alkanes can be processed by various suitable methods. The reaction mixture is preferably separated into a gas phase and a liquid phase.

Such a separation into a gas phase and a liquid phase can be effected, for example, by cooling the reaction mixture, to name only one of numerous suitable methods. In this case, for example, the mixture is cooled to a temperature of less than 60° C., preferably to about 40° C. to about 20° C., for example to about 30° C., and initially separated into a gas phase and a liquid phase.

The resulting product mixture of unreacted synthesis gas, alcohols, alkenes and alkanes can be cooled to lower temperatures of, for example, 150° C. or less, in particular to below 130° C., preferably to below 110° C. or to even lower temperatures of less than 80° C., for example about 40° C. to 20° C., especially to about 30° C., and separated into a gas phase and a liquid phase.

After separation into a gas phase and a liquid phase, the gas phase predominantly comprises the unreacted synthesis gas and any inert components present (e.g. nitrogen) and the methane formed as a by-product. The gas phase is usually recycled to the synthesis of the higher alcohols. Optionally, additionally, purification or conditioning of the gas phase, such as the conversion of the methane formed as a by-product to synthesis gas, is provided.

The liquid phase predominantly comprises the alcohols, alkenes and alkanes formed. By lowering the pressure, for example to less than 5 bar, in particular to ca. 1 bar, the alkenes and alkanes can be evaporated and separated off from the product mixture. Alternatively, the alkenes and alkanes can also be separated from the alcohols by other suitable methods.

For economic and/or ecological optimization of the process, it may be advantageous to convert the alkanes to synthesis gas, for example via partial oxidation, steam reforming or autothermal reforming, and to recycle them to the process. Optionally, the alkanes can also be dehydrogenated to the corresponding alkenes in order to increase the yield of alkenes. The alcohols remain in the liquid phase and, after separating off the water formed as co-product, are optionally marketed as a product mixture, for example as a fuel additive, or separated into the individual alcohols in a distillation process.

Alternatively, for example, the water can also already be separated off during the gas/liquid separation described above if the liquid partitions into an organic and an aqueous phase. The aqueous phase may also contain methanol and a little ethanol.

Depending on the respective composition and concentration of the products formed after the catalytic conversion of the synthesis gas, in the context of the process according to the invention, the aforementioned process parameters can be suitably varied or supplemented by further separation steps.

In addition to the downstream dehydration of the alcohols to form the alkenes, the value chain according to the invention also includes the direct integration of the consecutive dehydration of the alcohols into the process concept for the synthesis of the higher alcohols. There are several options for this and the process according to the invention therefore provides several alternative variants.

According to a first preferred variant of the process, the alkanes and alkenes are first separated off from the alcohols from the first mixture of alkanes, alkenes and alcohols obtained after the catalytic conversion of synthesis gas, and the alcohols separated off are then dehydrated.

In this variant, a mixture of separated alcohols can preferably first be separated into two or more fractions having different numbers of carbon atoms and only then can the respective individual fractions be dehydrated separately from one another in order to obtain the corresponding alkenes from the alcohols in the fractions in each case.

Furthermore, the mixture of alcohols can preferably be separated at least into a C2 fraction, a C3 fraction and a C4 fraction, and ethene, propene and butene can be obtained from these fractions. In this variant, the alkanes should preferably be separated off before the alcohols are dehydrated.

In this possible variant, the consecutive dehydration of the alcohols to form alkenes is carried out after the hydrocarbons have been separated off and after the alcohol mixture has been purified or separated into the respective pure alcohols. The separation of the alcohol mixture into the individual alcohols may be advantageous since it enables the individual alcohols to be dehydrated separately. Alcohols that are for example less suitable for the fuel market, alcohols that can be dehydrated under mild reaction conditions or inexpensively, or alcohols for which there is a corresponding alkene market can be dehydrated selectively to give the respective alkenes.

Alcohols for which a high price can be attained can be marketed directly. It is advantageous that the reaction conditions for the dehydration of the individual alcohols can be selected independently of one another. The disadvantage is that a separate plant for the dehydration is required for each alcohol or that the various fractions have to be dehydrated in batches.

According to a second preferred variant of the process according to the invention, the consecutive dehydration of the alcohols to form alkenes is carried out after the hydrocarbons have been separated off and before the alcohol-water mixture is separated into the individual alcohols.

In particular, the alkenes and alkanes can first be separated off from the first mixture, which is formed during the catalytic conversion of the synthesis gas and which comprises alcohols, alkenes and alkanes, and then a mixture comprising the alcohols of predominantly C2-C4 alcohols can then be dehydrated in the mixture to the corresponding alkenes. In contrast to the first variant mentioned above, the alcohols are not separated into the individual compounds having a different number of carbon atoms prior to the dehydration.

Optionally, after the dehydration, methanol and optionally water are separated off from the alkenes, and the alkenes are combined with the stream of alkenes and alkanes separated off before the dehydration. One possible option would be to separate a mixture of, for example, ethene, propene and butene into the individual alkenes without combining with another stream. A second alternative option would be to combine a mixture of, for example, ethene, propene and butene with the stream of alkenes and alkanes separated off before the dehydration and then to carry out further processing of the mixture of alkanes and alkenes, in which these are separated into a C2, a C3 and a C4 fraction and then the alkenes are each separated from the alkanes having the same number of carbon atoms.

In this process variant, the alkene mixture obtained by the dehydration can optionally be separated into individual alkenes, in particular into ethene, propene and butene. Thus, in this variant also, the pure alkenes can be obtained, which are suitable, for example, as starting materials for further syntheses, but the separation step into the individual compounds having a different number of carbon atoms takes place in this variant only after the dehydration, i.e. alkenes are separated from each other and not alcohols.

The separation of the alcohols from the alkenes and alkanes offers the possibility of carrying out the dehydration of the alcohols with a relatively pure reactant stream and as close as possible to the industrial processes for the dehydration of alcohols. It must be taken into account here that the industrial processes are optimized for the conversion of the individual alcohols and differ from one another in the choice of catalyst and the reaction conditions. For the conversion of the alcohol mixture, the reaction conditions must be selected in such a way that the conversion of all alcohols (with the exception of methanol) is made possible or the conversion of individual favored alcohols to the respective alkenes is at least favored.

Compared to the first variant, the dehydration of the alcohol mixture has the advantage that only one plant is required for the dehydration and a batchwise conversion can be avoided.

A third alternative preferred variant of the process according to the invention provides for carrying out the dehydration of the alcohols with the mixture of alkanes, alkenes and alcohols without the alcohols having been separated off from this mixture beforehand.

In this variant, methanol and optionally water are preferably removed from the resulting product mixture after the dehydration, after which the mixture of alkenes and alkanes can optionally be separated into two or more fractions, for example into C2, C3 and C4 fractions, and optionally the alkenes can then still each be separated from the alkanes having the same number of carbon atoms in the individual fractions, so that, for example, ethene, propene and butene are obtained as separate compounds in each case.

In this process variant, the removal of methanol and optionally water can be carried out by various suitable methods known per se to those skilled in the art. One possible option is to carry out the removal of methanol and water at a lower temperature and lower pressure than the preceding dehydration, preferably at a temperature in the range from 20° C. to 40° C. and a pressure of less than 5 bar, particularly preferably at a pressure of less than 2 bar.

Preferably, in this variant of the process, before the dehydration of the alcohols to the corresponding alkenes and after the catalytic conversion of the synthesis gas, at least one step is provided in which the product mixture obtained in this reaction is separated into a gas phase and a liquid phase, the liquid phase being used for the subsequent dehydration of the alcohols to the alkenes. Various methods are suitable for this gas/liquid separation.

By way of example only and not by way of limitation, the gas/liquid separation may be effected at a lower temperature and/or at approximately the same pressure as the preceding catalytic conversion of the synthesis gas.

In this variant of the process, the gas phase obtained in the separation is preferably at least partially recycled to the step of the catalytic conversion of the synthesis gas.

In this third possible variant of the process, the dehydration is thus carried out in the presence of the alkenes already formed and the alkanes. Carrying out the consecutive dehydration of the alcohols to alkenes after the gas-liquid separation offers the possibility of carrying out the dehydration at high pressures and mild reaction temperatures. However, the dehydration is carried out in the presence of the alkenes already formed and the alkanes. In this way, energy costs can possibly be reduced compared to the second alternative mentioned above. When selecting the respective reaction conditions for this process variant, it should be avoided that other components of the product mixture (alkenes, alkanes) react under the conditions of the catalytic dehydration or influence the dehydration of the alcohols.

A fourth possible alternative variant of the process according to the invention provides that after the catalytic conversion of the synthesis gas and after the subsequent dehydration of the alcohols to the corresponding alkenes, at least one step is provided in which the product mixture obtained in this reaction is separated into a gas phase and a liquid phase, methanol and optionally water then being separated off from the liquid phase and the alkanes being separated off.

In this variant of the process also, the gas phase obtained in the separation is preferably at least partially recycled to the step of the catalytic conversion of the synthesis gas.

In this variant of the process, the dehydration of the alcohols is thus carried out in the presence of the alkenes already formed, the alkanes and the unreacted synthesis gas. The temperature ranges at which the dehydration is carried out depend, inter alia, on the catalyst selected here. Since various catalysts are available, the temperature ranges are quite wide, for example from about 200° C. to about 400° C. It is therefore possible to select the reaction conditions for the dehydration of ethanol and propanol similar to those used in the synthesis of the higher alcohols by catalytic conversion of synthesis gas, so that in this variant it is possible and useful to carry out the dehydration of the alcohols directly in a reactor downstream of the catalytic synthesis of the alcohols from synthesis gas and without prior separation of the product mixture.

The dehydration is preferably carried out at a pressure of 1 bar to 100 bar.

The advantage here is that the product mixture does not have to be cooled and depressurized to a lower temperature and a low reaction pressure (for example 20 to 40° C., less than 5 bar, in particular about 1 bar), but can be reacted directly. In this way, the energy costs may possibly be reduced in comparison to the process variants 2 and 3 described above.

The various options for integrating the consecutive conversion of the alcohols to alkenes into the process concept for the catalytic synthesis of the higher alcohols from synthesis gas differ in each case in the composition of the reaction mixture and the prevailing process conditions, such as temperature and pressure. Integrating the dehydration of the alcohols into the process concept for the synthesis of the alkenes provides the opportunity to utilize the existing temperature and pressure levels of the alcohol synthesis for the dehydration.

The consecutive dehydration of the alcohols results in a targeted multi-stage synthesis of alkenes from synthesis gas having a substantially higher yield.

Compared to the catalytic processes for preparing alkenes, especially C2 to C4 alkenes, from synthesis gas that are hitherto known from the prior art, the two-stage synthesis according to the invention has the advantage of higher alkene yields. At this point, it should be mentioned that the two steps of the process according to the invention, namely the catalytic conversion of the synthesis gas to higher alcohols and the dehydration of the alcohols, can optionally also be carried out in the same reactor. Therefore, the term “two-stage” as used herein should not be understood to mean that two reaction steps must be carried out in separate reactors.

WO 2015/086151 A1 describes by way of example a process by which synthesis gas may be provided by purifying and conditioning various gas streams formed in a metallurgical works. Synthesis gas from such sources, for example, is suitable for the first process step described herein for the catalytic synthesis of alcohols having at least two carbon atoms from synthesis gas (also referred to herein as higher alcohols). However, all other suitable synthesis gas sources may in principle also be considered for the process according to the invention.

In the context of the present invention an overall process has been developed that allows preparation of alkenes (having two or more carbon atoms) in good yield starting from synthesis gas. The present application describes processes which, starting from the product mixture obtained during the reaction of synthesis gas, comprising carbon monoxide and/or carbon dioxide and hydrogen, offer economic, technological and/or ecological advantages over known processes, in particular over mere separation with subsequent individual marketing of the products/groups of substances. Particular attention was paid here to optimization of product separation in accordance with the synthesis steps. This relates, inter alia, to the respective physical process conditions (pressure, temperature) and the establishment of preferred/technically tolerable reactant ratios for the synthesis steps while taking into account economic boundary conditions in particular.

Due to the large plant capacities which are necessary for example for the utilization of significant quantities of metallurgical gas but also for other synthesis gas sources it is preferable to employ processes affording products having sufficiently large (potential) markets. It is therefore of particular interest to consider commodity chemicals employable for example in the plastics or fuels sectors.

According to the above description, four process variants in particular are preferred in the context of the present invention.

In variant 1, the process preferably comprises the steps of:
production of higher alcohols (having at least two carbon atoms) and of alkenes by catalytic conversion of synthesis gas;
separation of the resulting product mixture into a gas phase and a liquid phase;
separation of the alkenes and optionally of the alkanes formed as by-products from the alcohols obtained;
optionally separation of the alcohol mixture separated off from the alkenes and alkanes into individual compounds or groups of compounds, in particular ethanol, propanols, butanols and optionally methanol, with methanol and optionally water being separated off from the higher alcohols;
in each case separate dehydration of the previously obtained individual alcohols, in particular ethanol, propanol and butanol, the alkenes being obtained in each case.
optionally separating the alkene-alkane mixture, obtained after separating off the alcohols, into individual fractions having the same number of carbon atoms and separating the fractions into the respective alkenes and alkanes.

In variant 2, the process preferably comprises the steps of:
production of higher alcohols (having at least two carbon atoms) and of alkenes by catalytic conversion of synthesis gas;
separation of the resulting product mixture into a gas phase and a liquid phase;
separation of the alkenes and optionally of the alkanes formed as by-products from the alcohols obtained;
dehydration of the mixture of the alcohols previously separated off from the alkanes and alkenes, obtaining a mixture of alkenes, methanol and optionally water, methanol and water being separated off from the alkene mixture;
optionally combining the alkenes obtained after this separation with the alkenes and alkanes already previously obtained in the catalytic conversion of the synthesis gas;
separation of the resulting alkene-alkane mixture into individual compounds or groups of compounds, in particular ethene, propene, butene and optionally higher alkenes.

In variant 3, the process preferably comprises the steps of:
production of higher alcohols (having at least two carbon atoms) and of alkenes by catalytic conversion of synthesis gas;
separation of the resulting product mixture into a gas phase and a liquid phase;
dehydration of the previously obtained liquid phase product mixture comprising alcohols, alkenes and alkanes, wherein the alcohols in the mixture are dehydrated to the corresponding alkenes;
removal of methanol and optionally water from the product mixture;
separation of the resulting alkene-alkane mixture into individual compounds or groups of compounds, in particular ethene, propene, butene and optionally higher alkenes.

In variant 4, the process preferably comprises the steps of:
production of higher alcohols (having at least two carbon atoms) and of alkenes by catalytic conversion of synthesis gas;
dehydration of the resulting product mixture comprising alcohols, alkenes and alkanes, wherein the alcohols in the mixture are dehydrated to the corresponding alkenes;
separation of the resulting product mixture into a gas phase and a liquid phase;
removal of methanol and optionally water from the liquid phase;
optionally separation of the resulting alkene-alkane mixture into individual compounds or groups of compounds, in particular ethene, propene, butene and optionally higher alkenes.

In all four of the aforementioned process variants, at least partial recycling of the gas phase after the gas-liquid separation in the event of incomplete conversion is advantageous for the synthesis of higher alcohols.

The dehydration of the alcohols may be carried out not only by the aforementioned process variants but also by a combination of two or more of the aforementioned process variants. For example, the product mixture of higher alcohols (having at least two carbon atoms) and alkenes initially obtained by catalytic conversion of synthesis gas can be dehydrated predominantly to alkenes by process variant 4. If the conversion of the alcohols is not complete, the alcohols present in the liquid phase after the product mixture obtained has been separated into a gas phase and a liquid phase can be dehydrated to the corresponding alkenes, for example by means of one of process variants 1, 2 or 3. Ethanol, for example, the dehydration of which at 280° C. may not proceed completely and/or only slowly, could be dehydrated to ethene after separation of the product mixture obtained into a gas phase and a liquid phase by means of one of process variants 1, 2 or 3. In contrast to the dehydration of 1-propanol (ca. 200-250° C.), the industrial processes for the dehydration of ethanol are carried out at higher reaction temperatures of, for example, approximately 315-400° C. By combining process variants 1-4, for example, limitations can be circumvented that are not represented in the thermodynamic equilibrium.

Optionally, a combination of process variants 1-4 can also lead to advantages in the separation of the product mixture. Thus, it may be advantageous to separate individual fractions of alcohols and alkenes having the same carbon number preferably as alcohols rather than as alkenes from the product mixture of the conversion of synthesis gas to higher alcohols. For example, the removal of ethene from the product mixture of the higher alcohol synthesis during the separation of the resulting product mixture into a gas phase and a liquid phase is more complex than that of the long-chain alkenes, so that it can be advantageous to choose the reaction conditions for the dehydration according to process variant 4 in such a way that the ethanol, in contrast to the other alcohols, is not dehydrated, to separate the ethanol and to carry out the dehydration of the ethanol according to one of process variants 1, 2 or 3.

The provision of the synthesis gas for the catalytic conversion into alcohols according to the invention may comprise not only the preparation of the synthesis gas but also the purification and the conditioning of the synthesis gas. Both fossil fuels, such as natural gas, coal, but also CO-rich and CO₂-rich gases, for example from steel and cement works, and hydrogen can be used as feed. It is also possible to obtain the synthesis gas used from biomass. The hydrogen is preferably produced in a sustainable way with low CO₂ footprint, produced for example by means of water electrolysis or methane pyrolysis. The electricity for operating the hydrogen production is preferably provided using renewable energies.

In the context of a development of the present invention specific cobalt-containing catalysts combining properties of a methanol synthesis catalyst and a Fischer-Tropsch catalyst were developed. In this way the catalytic conversion of synthesis gas forms a product mixture which contains not only the higher alcohols (especially ethanol, propanol and butanol) but also high concentrations of hydrocarbons (especially C2-C4 alkenes and C1-C4 alkanes), water and CO₂.

Employed here is a catalyst which comprises grains of non-graphitic carbon having cobalt nanoparticles dispersed therein, wherein the cobalt nanoparticles have an average diameter dₚ in the range from 1 nm to 20 nm and the average distance D between individual cobalt nanoparticles in the grains of non-graphitic carbon is in the range from 2 nm to 150 nm and ω, the combined total mass fraction of metal in the grains of non-graphitic carbon is in the range from 30% by weight to 70% by weight of the total mass of the grains of non-graphitic carbon, wherein dₚ, D and ω satisfy the following relationship: 4.5 dp/ω<D≥0.25 dp/ω.

The process according to the invention particularly preferably employs a catalyst doped with a metal selected from Mn, Cu or a mixture thereof, wherein the grains of non-graphitic carbon have a molar ratio of cobalt to doped metal in the range from 2 to 15.

In experiments in the context of the present invention it has been found that the aforementioned grains of non-graphitic carbon having cobalt nanoparticles dispersed therein are obtainable from aqueous solutions of metallic precursors and organic carbon sources by combined spray drying or freeze drying of the aqueous solution and thermal treatment of the resulting intermediate at moderate temperatures.

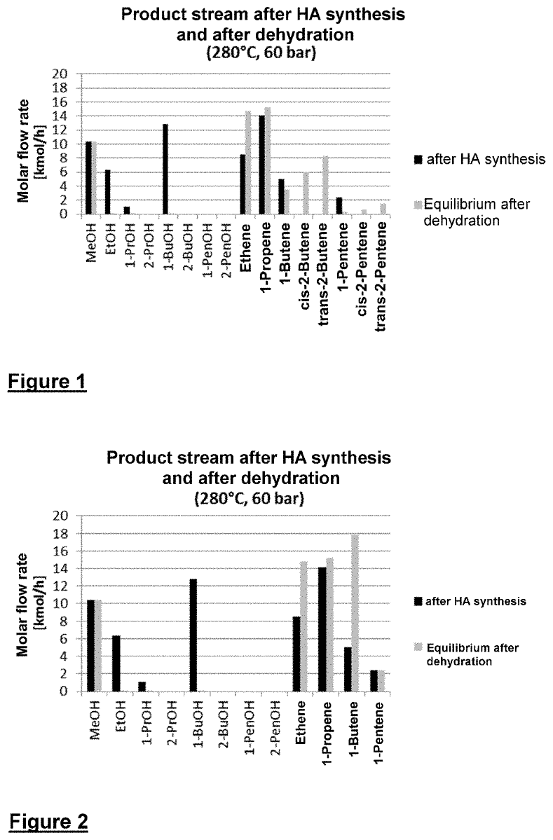

Non-graphitic carbon may be identified by those skilled in the art via TEM analysis (P W Albers, Neutron scattering study of the terminating protons in the basic structural units of non-graphitizing and graphitizing carbons, Carbon 109 (2016), 239-245, page 241, FIG. 1c).

Compared to present knowledge and also to the descriptions known from the literature, the aforementioned catalysts surprisingly have a significantly higher selectivity for alkenes than for alkanes (for example of the order of about 3:1). The product mixture obtained thus comprises not only the alcohols with the alkenes but also further products of value, the material rather than energetic utilization of which is advantageous from an economic and ecological standpoint.

In connection with an advantageous development of the invention a further important aspect is the separation of the products of value from the relatively complex product mixture at the reactor outlet. In addition to the products of value alcohols and alkenes, the product mixture may also contain residual gases (depending on the input gas: H₂, CO, CO₂, N₂) and by-products (especially alkanes, CO₂ and H₂O).

The present invention is described in more detail below on the basis of exemplary embodiments with reference to the accompanying drawings. In the figures:

FIG. 1 is a graphic representation of the product distribution after the catalytic conversion of synthesis gas to higher alcohols and subsequent dehydration of the product mixture at a temperature of 280° C. and a pressure of 60 bar, wherein the product distribution between the alcohols and the alkenes in thermodynamic equilibrium is shown under the assumption that an isomerization of the 1-alkenes to the 2-alkenes can take place.

FIG. 2 shows a graphic representation of the product distribution between the alcohols and the alkenes in thermodynamic equilibrium, assuming that no isomerization of the 1-alkenes to the 2-alkenes takes place.

For the working example shown in FIG. 1, the following equilibrium reactions were considered:

ethanol↔ethene+H₂O

1-propanol↔propene+H₂O

1-butanol↔1-butene+H₂O

1-pentanol↔1-pentene+H₂O

1-butene↔cis-2-butene

1-butene↔trans-2-butene

1-pentene↔cis-2-pentene

1-pentene↔trans-2-pentene

FIG. 1 shows the product distribution in thermodynamic equilibrium. According to this, after the first reaction step, the synthesis of the higher alcohols, the alcohols formed are predominantly ethanol and 1-butanol and the alkenes formed are predominantly 1-propene and 1-butene as well as some ethene and 1-pentene. After dehydration at 280° C., the main products in equilibrium are ethene and 1-propene, as well as the butenes trans-2-butene, cis-2-butene and 1-butene and some trans-2-pentene in decreasing proportions. This is due to the fact that in thermodynamic equilibrium, over a very long reaction time, trans- and cis-2-butene are formed from 1-butene, since these are thermodynamically more stable than 1-butene. However, if the residence time is shortened, it is possible to achieve that only, or at least predominantly, 1-butene is formed. Alcohols are practically no longer present, except for a small amount of methanol, which cannot be dehydrated and can be easily removed from the mixture.

Experiments and simulations on the catalytic synthesis of higher alcohols with subsequent dehydration according to the invention show that under the reaction conditions of the catalytic synthesis of higher alcohols, the dehydration of the alcohols to the alkenes is thermodynamically preferred (see FIG. 1). The synthesis of the higher alcohols was simulated on the basis of the experimental conversions and selectivities with the specific catalysts preferably used in this synthesis in the context of the present invention. The subsequent dehydration was calculated for an equilibrium reactor. The results clearly show that under the reaction conditions of the HA synthesis (280° C., 60 bar), a virtually complete conversion of the alcohols to the corresponding alkenes takes place. The results also show increasing formation of 2-butene and 2-pentene in thermodynamic equilibrium.

From a thermodynamic point of view, the dehydration of the alcohol mixture thus lends itself to the catalytic synthesis of higher alcohols at temperatures of ca. 280° C. The extent to which the dehydration actually proceeds under the reaction conditions may also depend on the respective catalysts used. It is also possible that other components of the product mixture (alkenes, alkanes, H₂, CO, CO₂) react under the conditions of the catalytic dehydration or affect the dehydration (e.g. also the C3+ alcohols) (see US 2009/0281362 A1). Among other things, these aspects depend on which of the aforementioned process variants is preferable in the individual case, for example, the in situ conversion of the alcohols to the corresponding alkenes may have advantages or disadvantages compared to a downstream dehydration, such as the dehydration of individual alcohols.

In the representation according to the diagram in FIG. 2, only the following equilibrium reactions are considered:

ethanol↔ethene+H₂O

1-propanol↔propene+H₂O

1-butanol↔1-butene+H₂O

1-pentanol↔1-pentene+H₂O

It is therefore assumed in FIG. 2 that no isomerization of the 1-alkenes to the 2-alkenes takes place. The diagram shows that dehydration of 1-butanol to 1-butene is possible. FIG. 2 thus represents the preferred product distribution in which no 2-alkenes are formed.

### EXAMPLE 1

Example 1 which follows specifies an exemplary product composition obtained in the catalytic conversion of synthesis gas by the process according to the invention using a catalyst which comprises grains of non-graphitic carbon having cobalt nanoparticles dispersed therein, wherein the cobalt nanoparticles have an average diameter dₚ in the range from 1 nm to 20 nm and the average distance D between individual cobalt nanoparticles in the grains of non-graphitic carbon is in the range from 2 nm to 150 nm and ω, the combined total mass fraction of metal in the grains of non-graphitic carbon, is in the range from 30% by weight to 70% by weight of the total mass of the grains of non-graphitic carbon, wherein dₚ, D and ω satisfy the following relationship: 4.5 dp/ω>D≥0.25 dp/ω. The catalyst used showed a high C2-C4 selectivity and alcohols, alkenes, and alkanes were formed. The CO selectivity in respect of the conversion to alcohols is about 28%, the CO selectivity in respect of the conversion to alkenes is about 32%. The precise selectivities of the catalytic conversion of the synthesis gas are apparent from table 1 which follows. The selectivities reported in table 1 were normalized to the products detected in the catalytic tests (C1-C5 alcohols, C1-C5 alkenes and C1-C5 alkanes, CO₂). The analysis of the CO conversion indicates that, in addition to the specified products detected, long-chain C6+ alcohols, C6+ alkenes and C6+ alkanes, and in some cases aldehydes, are also formed.

TABLE 1

| | CO selectivity [%] |
|---|---|
| CO₂ | 9.8 |
| Methane | 17.9 |
| Ethane | 4.6 |
| Propane | 4.3 |
| Butane | 3.0 |
| Pentane | 0.3 |
| Ethene | 6.0 |
| 1-Propene | 15.1 |
| 1-Butene | 7.2 |
| Pentene | 4.2 |
| Methanol | 3.7 |
| Ethanol | 4.6 |
| 1-Propanol | 1.1 |
| 2-Propanol | 0.0 |
| 1-Butanol | 18.3 |
| Alkanes (C2-C5) | 12.2 |
| Alkenes (C2-C5) | 32.5 |
| Higher alcohols | 24.0 |

This example employed a pulverulent catalyst. The catalyst may alternatively also be pressed into tablets for example.

Table 1 above shows that the catalytic conversion of synthesis gas according to the invention affords a relatively high CO selectivity for the alcohols and for the alkenes. In comparison, the selectivity for the alkanes is lower. The higher alcohols (from C2) can be converted to further alkenes in the subsequent dehydration step, so that, including this dehydration step, in total the synthesis gas can be converted to alkenes with a CO selectivity of about 56%, for example, wherein the 1-alkenes are preferably obtained (see above) in the dehydration, so that 1-propene, 1-butene and some 1-pentene are formed in addition to ethene (see FIG. 2).

### EXAMPLE 2

A possible process for separating the product mixture obtained in the catalytic conversion of synthesis gas is described below by way of example. The exemplary separation process described below is preferably used for process variants 1 and 2 and describes the separation of the mixture of alcohols, alkenes and alkanes obtained by the reaction of the synthesis gas from the gas phase and its subsequent separation into a mixture of alcohols and a mixture of hydrocarbons. When using variants 3 or 4, individual steps of this process can be adapted to the product mixture obtained after the conversion or omitted due to the previous conversion of the product mixture.

### Inert Gas Removal

Catalytic conversion of a synthesis gas stream under the conditions of the process according to the invention affords a product stream at a temperature of 280° C. and a pressure of 60 bar. This is initially decompressed to a pressure of 5 to 20 bar, preferably to about 10 bar, in a turbine to generate electrical energy which may be used for the power requirements of the process.

The subsequent gas-liquid separation, which serves in particular to separate the inert gases (for example nitrogen) and unreacted components of the synthesis gas (hydrogen, carbon monoxide, carbon dioxide and methane), is carried out by absorbing the product stream in a diesel oil (reference component dodecane) or alternatively in an alkane or a hydrocarbon mixture with a comparatively low viscosity of, for example, less than 10 mPas at room temperature and preferably with a comparatively high boiling point of, in particular, more than 200° C. The water is not absorbed in the process, but is largely condensed as the second liquid phase.

The two liquid phases (organic phase and aqueous phase) can then be separated in a decanter, the hydrocarbons barely, but the alcohols partially, passing into the aqueous phase. The alcohols may be distilled out of the water again as azeotropes by means of a first column for example. Alcohols and hydrocarbons are then desorbed from the diesel oil, which may be done in a column. The diesel oil may be recycled into the absorption process after desorption. At relatively low inert gas fractions in the product stream of the catalytic conversion of synthesis gas, a condensation of the low-boiling components may alternatively also be considered.

### Separation of Alcohols/Hydrocarbons

The subsequent separation of alcohols and hydrocarbons is carried out by distillation in a second column, preferably at a high pressure of 10 bar to 40 bar for example, in order that the C3 fractions remain condensable even in the presence of any residues of inert gas. This separation is preferably carried out such that the hydrocarbons are practically completely removed from the alcohol fraction at the column bottom, while smaller alcohol contents (in particular methanol) in the hydrocarbons may be tolerated. This process may optionally be assisted by a solubility-based membrane.

### Preparation of the Hydrocarbons

In a third distillation column the hydrocarbons are obtained overhead at elevated pressure of for example 5 bar to 20 bar while the remaining water and the alcohols dissolved therein are obtained in the bottoms and separated. This stream can be recycled to the first distillation column to recover the alcohols. The condenser of the column may be a partial condenser for example. The outputs of the column are a gas phase of hydrocarbons and inerts, a liquid phase of hydrocarbons and an aqueous phase which may be returned to the column as reflux.

### Dewatering of the Alcohol Fraction

The alcohol fraction may have a water content of about 10% for example. This water may be removed using a molecular sieve for example.

A contemplated alternative method for removing the water from the alcohol fraction is extractive distillation for example with ethylene glycol, though this requires a further separation step since the water is pulled into the bottoms by the ethylene glycol while the alcohols methanol and ethanol proceed overhead practically free from water. About half of the propanol and all of the butanol remain in the bottoms and these C3-C4 alcohols must likewise be removed from the ethylene glycol overhead in a subsequent column.

A third suitable alternative is pervaporation. Water passes selectively through a membrane and is withdrawn in vaporous form as permeate. Energy consumption is even lower than for a molecular sieve.

A further alternative method would be an azeotropic distillation, for example with butane or pentane as a selective additive.

1.-18. (canceled)

19. A process for preparing alkenes by catalytic conversion of synthesis gas to give a first mixture comprising alkenes and alcohols, wherein alcohols present in the first mixture are converted to corresponding alkenes in at least one subsequent step by dehydration, wherein at least one alkene having two to four carbon atoms is obtained as an isolated product from a product mixture by processing thereof and/or separation steps, either before or after the dehydration of the alcohols.

20. The process of claim 19 wherein from the first mixture of alkenes, alcohols, and alkanes obtained after the catalytic conversion of synthesis gas, the alkanes and alkenes are separated from the alcohols first and then the separated alcohols are dehydrated.

21. The process of claim 20 wherein a mixture of separated alcohols is first separated into two or more fractions having a different number of carbon atoms and only then is at least one individual alcohol fraction dehydrated to obtain the corresponding alkene from the alcohol.

22. The process of claim 21 wherein the mixture of alcohols is separated at least into a C2 fraction, a C3 fraction, and a C4 fraction and at least one of ethene, propene, or butene is obtained from one or more of these fractions.

23. The process of claim 20 wherein after the alkenes and alkanes have been separated off, a mixture of predominantly C2-C4 alcohols comprising the alcohols is then dehydrated in the mixture to form the corresponding alkenes.

24. The process of claim 19 wherein after the dehydration, methanol is separated off from the alkenes and the alkenes are combined with a stream of alkenes and the alkanes separated off prior to the dehydration, wherein an alkene-alkane mixture is separated into individual compounds or compound groups that include fractions each having the same number of carbon atoms, including C2 or C3 or C4 hydrocarbons.

25. The process of claim 24 wherein the alkenes are each separated off from the alkanes from individual fractions, each having the same number of carbon atoms, so that ethene, propene, and butene are obtained.

26. The process of claim 19 wherein the dehydration is performed at temperatures in a range from 200° C. to 400° C. and/or at a pressure from 1 bar to 100 bar.

27. The process of claim 19 wherein the dehydration of the alcohols is performed with the first mixture of alkanes, alkenes, and alcohols without the alcohols having been separated off from the first mixture beforehand.

28. The process of claim 27 wherein after the dehydration, methanol is separated off from a product mixture obtained, after which a remaining mixture of alkanes and alkenes is separated into individual fractions each having the same number of carbon atoms, including C2, C3, or C4 fractions, and the alkene is separated off from the alkane from at least one of the individual fractions each having the same number of carbon atoms, so that at least one of ethene, propene, or butene is obtained.

29. The process of claim 24 wherein the methanol is removed at a lower temperature and a lower pressure than the dehydration.

30. The process of claim 19 wherein before the dehydration and after the catalytic conversion of the synthesis gas, the process comprises separating a product mixture obtained in the catalytic conversion into a gas phase and a liquid phase, wherein the liquid phase is used for subsequent dehydration of the alcohols to the alkenes.

31. The process of claim 30 wherein the gas phase is at least partially recycled to the catalytic conversion of the synthesis gas.

32. The process of claim 19 wherein after the catalytic conversion of the synthesis gas and after subsequent dehydration of the alcohols to corresponding alkenes, the process comprises separating a product mixture obtained in the catalytic conversion into a gas phase and a liquid phase, wherein methanol is then separated off from the liquid phase.

33. The process of claim 32 wherein the gas phase is at least partially recycled to the catalytic conversion of the synthesis gas.

34. The process of claim 32 wherein after the catalytic conversion of the synthesis gas the product mixture is processed by steps comprising:
at least partially absorbing the alkenes and alcohols in a high-boiling hydrocarbon or hydrocarbon mixture as an absorption medium;
separation of gases not absorbed into the absorption medium as a gas phase;
separating an aqueous phase from an organic phase of the absorption medium; and
desorption of the alkenes, the alkanes, and the alcohols from the absorption medium.

35. The process of claim 19 wherein using a catalyst in the catalytic conversion of the synthesis gas, wherein the catalyst comprises grains of non-graphitic carbon having cobalt nanoparticles dispersed therein, wherein the cobalt nanoparticles have an average diameter dₚ in a range of 1 nm to 20 nm, wherein an average distance D between individual cobalt nanoparticles in the grains of non-graphitic carbon is in a range of 2 nm and 150 nm, wherein a combined total mass fraction ω of metal in the grains of non-graphitic carbon is in a range from 30% by weight to 70% by weight of a total mass of the grains of non-graphitic carbon, wherein 4.5 dₚ/ω>D≥0.25 dₚ/ω.

36. The process of claim 35 wherein a material of the catalyst is doped with a metal selected from Mn, Cu, or a mixture thereof, wherein the grains of non-graphitic carbon have a molar ratio of cobalt to doped metal in a range of 2 to 15.
